(19) United States
(12) Patent Application Publication
(10) Pub. No.: US 2020/0023025 A1
(43) Pub. Date: Jan. 23, 2020

(54) NUTRACEUTICAL COMPOSITIONS FOR DYSMETABOLIC DISEASES

(71) Applicant: Lorena Angela Assunta CAPOCCIA, Codogno (IT)

(72) Inventor: Lorena Angela Assunta CAPOCCIA, Codogno (IT)

(21) Appl. No.: 16/496,837

(22) PCT Filed: Mar. 19, 2018
(86) PCT No.: PCT/IB2018/051814

§ 371 Date: Sep. 23, 2019

(30) Foreign Application Priority Data
Mar. 22, 2017 (IT) 10207000031593

(51) Int. Cl. A61K 36/185; A61K 36/064; A61K 35/742; A61K 36/8962; A61K 31/131; A61K 31/4415; A61K 31/714; A61K 31/519; A61K 31/341; A61K 9/06; A61K 9/00

(52) U.S. Cl. CPC A61K 36/185; A61K 36/064; A61K 35/742; A61K 36/8962; A61K 31/131; A61K 9/0053; A61K 31/714; A61K 31/519; A61K 31/341; A61K 9/06; A61K 31/4415

(57) ABSTRACT

The present invention relates to a composition comprising: extract of flowers, leaves, roots and/or seeds of Nelumbo nucifera; Saccharomyces, preferably selected from S. cerevisiae and S. boulardii; Bacillus coagulans; mineral salts; group B and C vitamins; optionally garlic, optionally taurine. The use of said composition in the prevention and/or treatment of the dysmetabolic syndrome is also claimed.

The present invention relates to a composition of micronutrients selected from the group comprising: extract of flowers, leaves, roots and/or seeds of Nelumbo nucifera, Saccharomyces, preferably selected from S. cerevisiae and S. boulardii; Bacillus coagulans; mineral salts; group B and C vitamins; optionally garlic, optionally taurine.

The use of said composition in the prevention and/or treatment of the dysmetabolic syndrome is also claimed.

### BACKGROUND ART

Metabolic syndrome (MS) is a group of risk factors such as obesity, impaired glucose metabolism, high blood pressure, hypertriglyceridemia, hypercholesterolemia, which may increase the likelihood of incurring cerebrovascular diseases (heart attack and cerebral ischemia) and diabetes. Metabolic syndrome is a very common disease: it is estimated that about 25% of the world population is affected.

According to the WHO, noncommunicable diseases (NCDs), which include cardiovascular diseases, cancer, diabetes and chronic respiratory disorders, cause 70% of deaths worldwide.

The number of diabetics is very high and constantly growing. Data collected by Istituto Superiore della Sanità report that in Italy 10% of men and 7% of women are diabetic; according to the WHO, there are about 400 million people with diabetes worldwide. Cardiovascular diseases (CVD) also afflicts a significant proportion of the world's population and related mortality is very high. In fact, cardiovascular diseases are the main cause of death in the world. In Italy, 44% of total deaths are due to CVD; in Europe, they account for 48% of all deaths (4.3 million people). People who overcome the acute cardiac event become chronically ill.

Moreover, according to the WHO, the reduction of the main risk factors also obtained by adopting correct eating habits and physical activity is a big weapon in the prevention of NCDs and in particular of MS.

The lifestyle of the populations of high-income countries has profoundly changed since the late 1960s and is characterized by a progressive reduction in energy needs, combined with the tendency to eat more than necessary in an unbalanced way. In addition, frequently, the foods taken have small amounts of micro-nutrients due to production and storage processes.

The use of food supplements has been widespread for years. A food supplement cannot compensate for inappropriate eating habits but, if necessary, it integrates a varied and balanced diet.

The Lotus plant, Nelumbo nucifera Gaertner, is born spontaneously in South Korea and in Japan. It is a perennial plant of the Paleozoic era, belonging to the Ninfeacee family. The different parts of the plant have shown diverse effects such as improvement of learning and memory, hepatoprotective effect, anti-obesity effect, anti-HIV effect, anti-tumor effect, diuretic activity, antipyretic activity, antidepressant effect, anti-inflammatory activity and antidepressant effect. Moreover, having the ability to reduce external heat, maintain moisture inside the body and support hemostasis, the leaves of Nelumbo nucifera have been used for the treatment of diarrhea, edema and for the disorders of hemostasis and coagulation occurring in hot and humid conditions. Nuciferine (1,2-dimethoxy-6a-β-aporphine) and Quercetin (3,3′,4′,5,7-pentahydroxyflavone) are contained in the lotus flower. The root of the lotus is characterized by its richness in phytonutrients, minerals, especially copper, iron, zinc, magnesium and manganese, and vitamins.

Chang Ho et al. (Antioxidant and neuronal cell protective effects of aqueous extracts from lotus leaf tea. J. Agriculture and Life Science 2012 46(2):115-127) describe positive effects obtained with lotus leaf extract in the treatment of obesity and hyperlipidemia in rats, especially when used in combination with taurine.

Kalpana S, Neelam C (Effect of Nelumbo nucifera aqueous leave and flowers extract on lipid profile of alloxan induced diabetic albino rats. Intl J Institutional Pharmacy and Life Sciences. Volume 6) report that the extracts of lotus leaves and flowers play a positive role in the treatment of diabetes and play a cardio-protective role in rats in which diabetes has been induced by alloxan.

Probiotics are living organisms which are beneficial to health if ingested. Probiotics are essentially used to counteract a number of conditions such as diarrhea, candida, urinary tract infections, food intolerances. There is an extensive literature analyzing the role of probiotics in dysmetabolism, however some meta-analyzes have shown poor probiotic activity in the case of subjects with high blood cholesterol and blood sugar levels.

Magnesium is an oligoelement involved in the metabolism of carbohydrates and amino acids and which facilitates the use of B, E and C vitamins. It is considered a bivalent ion fundamental for living organisms, involved in over 300 biochemical reactions in the human body.

Garlic, due to allicin, has a proven antidyslipidemic, anti-atherosclerotic and platelet antiaggregant effect. Furthermore, garlic has an effect on blood pressure, presumably due to the activation of the production of nitrogen monoxide which exerts a vasodilator action. Garlic also has an important antiseptic and antimicrobial effect.

B vitamins reduce the concentration of homocysteine in blood.

Vitamin C supplementation is capable of decreasing fasting blood glucose, insulin, glycosylated hemoglobin, total cholesterol, LDL cholesterol and triglycerides.

Since the risk factors underlying MS are multiple and in close correlation, it is necessary to counter them simultaneously, continuously and synergistically.

It is object of the present invention to provide an integrating agent capable of operating simultaneously on blood glucose, arterial pressure, triglycerides and cholesterol.

### DETAILED DESCRIPTION OF THE INVENTION

The invention relates to a composition capable of preventing and treating the dysmetabolic syndrome, i.e. of intervening in hyperglycemia, hypertension, hypercholesterolemia, triglyceridemia and excess body weight.

The composition comprises:

extract of flowers, leaves, roots and/or seeds of lotus, Nelumbo nucifera;
mineral salts, preferably magnesium;
probiotics selected from the group comprising: Saccharomyces, Bacillus coagulans;
group B and/or C vitamins.

In a further embodiment, said composition also comprises garlic.

In a further embodiment, said composition also comprises taurine.

The composition according to the present invention has surprisingly proved capable of promoting assimilation of the vitamins and minerals taken through food and contained in the composition of the present invention.

The probiotic Saccharomyces has been selected herein as it is a rich and complete source of B vitamins, selenium, chromium, glutathione, sulfur, essential amino acids, phosphorus, potassium and magnesium. It also contains vanadium, chrome, molybdenum and nickel. The cellular membrane of Saccharomyces is very rich in β-glucans and in particular of β-(1, 3) or (1, 4) glucans.

Bacillus coagulans is extremely resistant, where it resists to the external environment, to the gastric acid pH and to the basic intestinal pH and survives many handling processes, such as food cooking, due to its ability to form endospores. This probiotic has a high capacity to adhere to the gastro-intestinal mucosa. Its presence ensures greater production of lactic acid necessary to maintain the balance of the gastrointestinal bacterial flora, in addition to metabolizing glucose.

In one embodiment, said composition comprises:
extract of flowers, leaves, roots and/or seeds of Nelumbo nucifera between 0.4% and 30% w/w;
Saccharomyces, preferably selected from S. cerevisiae and S. boulardii, in amounts of about 1×10⁸ to 1×10¹² cells/ml of total composition;
Bacillus coagulans, in amounts of about 1×10⁷ to 1×10¹² cells/ml of total composition;
Mineral salts in amounts of about 1% to about 50% w/w;
Vitamin B6 in amounts of about 0.1% to about 3% w/w;
Vitamin B9 in amounts of about 5×10⁻⁴% to 10×10⁻³% w/w;
Vitamin B12 in amounts of about 2×10⁻²% to 0.4% w/w;
Vitamin C in amounts of about 0.2% to 13% w/w;
Garlic in amounts of about 0% to 90% w/w;
Taurine in amounts of about 0% to 90% w/w.

In a preferred embodiment, said composition comprises:
extract of flowers, leaves, roots and/or seeds of Nelumbo nucifera between 10 and 20% w/w;
Saccharomyces, preferably selected from S. cerevisiae and S. boulardii, in amounts of about 1×10⁹ to 1×10¹¹ cells/ml of total composition;
Bacillus coagulans, in amounts of about 1×10⁸ to 1×10¹⁰ cells/ml of total composition;
Magnesium in amounts of about 10% to about 30% w/w;
Vitamin B6 in amounts of about 0.8% to about 2% w/w;
Vitamin B9 about 0.001% w/w;
Vitamin B12 in amounts of about 0.01% to 0.4% w/w;
Vitamin C in amounts of about 1% to 9% w/w;
Garlic in amounts of about 0% to 40% w/w;
Taurine in amounts of about 0% to 40% w/w.

In a further embodiment, said composition comprises:
extract of flowers, leaves, roots and/or seeds of Nelumbo nucifera between 12 and 17% w/w;
Saccharomyces, preferably selected from S. cerevisiae and S. boulardii, about 1×10⁹ cells/ml of total composition;
Bacillus coagulans, about 10⁸ cells/ml of total composition;
Magnesium in amounts of about 15% to about 20% w/w;
Vitamin B6 in amounts of about 1.2% to about 1.7% w/w;
Vitamin B9 about 0.001% w/w;
Vitamin B12 in amounts of about 0.08% to 0.2% w/w;
Vitamin C in amounts of about 3% to 8% w/w;
Garlic in amounts of about 0% to 40% w/w;
Taurine in amounts of about 0% to 40% w/w.

In a preferred embodiment, said composition comprises, on 1 g of the total preparation: 1 to 4 mg of lotus flower extract, 1*10\hat{ }⁹ to 9*10\hat{ }⁹ cells of Saccharomyces cerevisiae, 1*10\hat{ }⁸ to 9*10\hat{ }⁸ cells of Bacillus coagulans, 80 to 200 mg Magnesium, 0.05 to 0.30 mg Vitamin B6, about 0.0001 mg Vitamin B9, 0.005 to 0.05 mg Vitamin B12, 0.1 to 1 mg Vitamin C, excipients as needed to 1 g.

A further aspect of the present invention is a formulation to be used as a food supplement which comprises the composition according to the present invention. Typically, said composition is in the form of a chewable tablet or a tablet which disintegrates in the mouth, liquid, powder, powder contained in a sachet, soft gelatin capsule, or hard gelatin capsule. In some embodiments, the polymers of the invention are provided as pharmaceutical compositions as in the form of liquid compositions. In various embodiments, the pharmaceutical composition contains a polymer dispersed in a suitable liquid excipient. Suitable liquid excipients are known in the art; see, for example, Remington's Pharmaceutical Sciences. In a preferred embodiment, the formulation is a hard capsule.

Typically, each capsule contains from 150 to 400 mg of Nelumbo nucifera, or from 200 to 350 mg, or 300 mg.

In a preferred embodiment, said pharmaceutical formulation is in capsule form. Said formulation is taken from 1 to 10 times a day, preferably from 2 to 5 times a day, even more preferably 3 times a day for use in the prevention and/or treatment of the dysmetabolic syndrome.

The present invention further relates to a method for preventing and/or treating the dysmetabolic syndrome, where said method comprises administering the composition described in the present invention. In a preferred embodiment, said administration is topical.

In a further embodiment, the composition has a topical use. It is then formulated with excipients known to those skilled in the art in the form of a gel, cream or ointment.

The formulation for topical use according to the present invention has proved to be particularly advantageous, where the composition surprisingly maximizes the bioavailability of β-glucans, delivered therein by probiotics. Furthermore, the composition according to the present invention maximizes the bioavailability of active ingredients useful for maintaining the good health of skin, hair, nails and teeth.

The surprising effects of the composition according to the present invention are explained by:
Availability directly in the host intestine of the active ingredients in high doses which promote the increase in growth rate, vitality and survival of probiotics.
Permanence over time of the active ingredients in the host intestine, obtained due to the probiotics.
Synergistic effect of the active ingredients which form the composition, where the proliferation of probiotics and the absorption of the active ingredients are promoted.
Administration of the composition according to the present invention does not lead to the onset of side effects.

Surprisingly, the composition according to the present invention has proved to be useful in maintaining the integumentary apparatus and cutaneous appendages (hair, nails and hair), also showing precious dermatological actions in improving pathologies such as seborrhea and acne and the effects of psoriasis on the skin.

### EXAMPLES

Example 1: preparation and characterization of lotus leaf extract Lotus leaves are extracted by methods known in the art, for example with a mixture of methyl alcohol and 1% hydrochloric acid (50:50, v/v) by sonication for 15 minutes. After centrifugation, the supernatant is filtered and subjected to liquid chromatography. The sample comprises:
N-nornuciferine: from 0.82±0.02 mg/g to 1.43±0.01 mg/g
O-nornuciferine: from 2.35±0.01 mg/g to 4.47±0.01 mg/g
Nuciferine: from 4.83±0.03 mg/g to 7.61±0.04 mg/g
Remerine: from 0.31±0.01 mg/g to 0.62±0.01 mg/g

Example 2: preparation of a composition according to the present invention.

1.5 mg of lotus flower extract, obtained as per example 1, are mixed with about 2*10\hat{ }⁹ cells of Saccharomyces cerevisiae, about 10\hat{ }⁸ cells of Bacillus coagulans, 120 mg Magnesium, 0.15 mg Vitamin B6, 0.0001 mg Vitamin B9, 0.01 mg Vitamin B12, 0.5 mg Vitamin C. The composition is increased to 1 g with excipients known in the art.

Example 3: use of the composition according to the present invention

The formulation according to example 2 is administered 3 times a day to 50 patients with MS for a prolonged period of 3 months. Symptom remission was observed in 75% of subjects treated.

1. A composition comprising:
an extract of flowers, leaves, roots and/or seeds of Nelumbo nucifera;
Saccharomyces;
Bacillus coagulans;
one or more mineral salts;
group B and C vitamins;
optionally garlic; and
optionally taurine.

2. The composition according to claim 1, comprising:
the extract of flowers, leaves, roots and/or seeds of Nelumbo nucifera in an amount between 0.4% and 30% w/w;
the Saccharomyces in an amount of between about 1×10⁸ and 1×10¹² cells/ml of total composition;
the Bacillus coagulans, in an amount of between about 1×10⁸ and 1×10¹² cells/ml of total composition;
the one or more mineral salts in an amount of between about 1% and about 50% w/w;
Vitamin B6 in an amount of between about 0.1% and about 3% w/w;
Vitamin B9 in an amount of between about 5×10⁻⁴% and 10×10⁻³%w/w;
Vitamin B12 in an amount of between about 2×10⁻²% and 0.4% w/w;
Vitamin C in an amount of between about 0.2% and 13% w/w;
the garlic in an amount of between about 0% and 90% w/w; and
the taurine in an amount of between about 0% and 90% w/w.

3. The composition according to claim 1 comprising:
the extract of flowers, leaves, roots and/or seeds of Nelumbo nucifera in an amount between 10 and 20% w/w;
the Saccharomyces, in an amount of between about 1×10⁹ and 1×10¹¹ cells/ml of total composition;
the Bacillus coagulans, in an amount of between about 1×10⁸ and 1×10¹⁰ cells/ml of total composition;
magnesium in an amount of between about 10% and about 30% w/w;
Vitamin B6 in an amount of between about 0.8% and about 2% w/w;
Vitamin B9 in an amount of about 0.001% w/w;
Vitamin B12 in an amount of between about 0.01% and 0.4% w/w;
Vitamin C in an amount of between about 1% and 9% w/w;
garlic in an amount of between about 0% and 40% w/w; and
taurine in an amount of between about 0% and 40% w/w.

4. The composition according to claim 1, comprising:
1 to 4 mg of lotus flower extract,
1×10⁹ to 9×10⁹ cells of the Saccharomyces cerevisiae,
1×10⁸ to 9×10⁸ cells of the Bacillus coagulans,
80 to 200 mg magnesium,
0.05 to 0.30 mg Vitamin B6,
about 0.0001 mg Vitamin B9,
0.005 to 0.05 mg Vitamin B12,
0.1 to 1 mg Vitamin C, and
excipients,
based on 1 g of the composition.

5. A method of treating a dysmetabolic disease, the method comprising administering the composition of claim 1 to a subject in need thereof.

6. A method of maintaining the tegumentary apparatus and skin structures, body hair, nails and hair, the method comprising administering the composition of claim 1 to a subject in need thereof.

7. A method for treating seborrhea, acne, and/or psoriasis, the method comprising administering the composition of claim 1 to a subject in need thereof.

8. A pharmaceutical formulation comprising the composition according to claim 1.

9. The pharmaceutical formulation according to claim 8, in the form of gel, cream or ointment.

10. The pharmaceutical formulation according to claim 8, further comprising one or more excipients for oral administration.

11. A method of treating a dysmetabolic syndrome, the method comprising administering the pharmaceutical formulation of claim 8 to a subject in need thereof 1 to 10 times daily.
